(19) United States
(12) Patent Application Publication
(10) Pub. No.: US 2011/0288449 A1
(43) Pub. Date: Nov. 24, 2011

(54) Method of lysing a thrombus

(76) Inventor: Jan-Peter Schenkengel, Magedeburg (DE)

(21) Appl. No.: 12/381,412

(22) Filed: Mar. 10, 2009

(51) Int. Cl. A61N 7/00

(52) U.S. Cl. 601/2

(57) ABSTRACT

A thrombolytic method of dissipating a thrombus by an application of ultrasound of a frequency in a diagnostic range in combination with a microbubble contrast agent.

### BACKGROUND OF THE INVENTION

1. Field of the Invention

The invention, in general, relates to a novel method of lysing or dissipating a thrombus and, more particularly, of dissipating a thrombus by an application of a contrast agent in combination with ultrasonic energy.

2. The Prior Art.

The occlusive arterial disease known as thrombosis is a not uncommon and serious vascular disease. It is particularly prone to occur in the lower extremities and often defies easy diagnostic detection. It results from blood clotting in natural as well as artificial veins. Depending upon the severity of the alteration in the various vascular ranges, a variety of operating techniques have been developed for the removal of a thrombus, such as, for instance, surgically formed artificial and venous arterial bypasses in addition to pharmaceutical intervening or prophylactic treatments including, inter alia, intravascular lysogenic treatments on the bases of rtPA, streptokinase or urokinase. Quite often, the lysogenic medication must be administered in very high doses which may, however, lead to uncontrollable and even lethal hemorrhages in all sections of the body. In this connection, it is clinically known that the complications increase exponentially with increased doses and the duration of the treatment.

One way of increasing the effect of lysogenic treatments is by mechanically inducing an increase in the surface of a thrombus. In this manner, the lysogenic medication can affect a larger surface of the thrombus and, in turn, result in its increased effectiveness and a more effective lysogenic treatment of a thrombus. It is known that the surface of a thrombus can be mechanically increased by an application ultrasound in the presence of an ultrasound contrast agent.

Ultrasound contrast agent of the kind of interest in this context usually consists of small bubbles of sulfurhexafluoride or dodecafluoropentane, hereafter referred to as microbubbles, encased in a membrane of a lipid layer. The difference in impedance between blood and micro bubbles yields an increased sonar reflection which, in turn, results in increased power of an ultrasound signal. The ultrasound causes the micro bubbles to oscillate, and the oscillations lead to an eventual collapse or implosion of the micro bubbles. As a result, mechanical energy of pressure and temperature of a significantly higher order of magnitude than those of the atmosphere are released and cause partial or complete mechanical destruction of the thrombus.

Thus, there has been no lack of attempts to devise reliable thrombolytic treatments based upon ultrasound in combination with contrast agents. For instance, U.S. Pat. No. 5,695,460 issued to Siegel et al. on 9 Dec. 1997 discloses a method of applying a combination of ultrasonic energy and certain ultrasound imaging agents. It aims at dissolving arterial thrombi by ultrasonically radiating a micro bubble-containing echo contrast agent, proximate the thrombosis vessel. It is said that the ultrasound may be applied intravascularly, by means of a miniature transducer, or by a guide wire for transmitting ultrasound directly into the vessel, or transcutaneously by an external generator and transducer, the ultrasound being in the range of from 24 kHz to 53 kHz.

Experiments conducted in this area with and without a contrast agent have generally been restricted to low-frequency ultrasound in a range of from about 10 kHz to about 50 kHz. Without a contrast agent, experiments have also been carried out with ultrasound in the 1 MHZ range. The power of the ultrasound applied was, however, at a level significantly higher than the level of diagnostic ultrasound. Presumably, the high ultrasonic power in the early experiments has been the reason for the frequent occurrence of hemorrhages and, last but not least, for the termination of such experiments, particularly in the context of cerebral studies.

### OBJECTS OF THE INVENTION

Accordingly, it is an object of the invention to provide for an ultrasonic treatment yielding useful thrombolytic effects without detrimental effects to a patient.

A more specific object of the invention is to provide a method of treating a thrombus with ultrasound with and without a contrast agent but without the risk of causing excessive hemorrhaging in the patient.

Still another object of the invention is to provide a treatment of the kind referred to which can be practiced with conventional diagnostics equipment.

### BRIEF SUMMARY OF THE INVENTION

In the accomplishment of these and other objects the invention provides a thrombolytic treatment involving an application of ultrasonic energy in the range of from about 1 MHZ to about 20 MHZ in the presence of an ultrasonic contrast agent. Preferably, the contrast agent is of the kind containing gaseous micro bubbles, such as, for instance, the contrast agent marketed by Bracco Diagnostics, Inc. of Plainsboro, N.J. under its trademark SonoVue®.

### DESCRIPTION OF THE DRAWINGS

The novel features which are considered to be characteristic of the invention have been set forth with particularity in the appended claims. The invention itself, however, in respect of its structure, construction and lay-out as well as manufacturing techniques, together with other objects and advantages thereof, will be best understood from the following description of preferred embodiments when read in connection with the appended drawings, in which:

FIG. 1 schematically shows a model of a pump-driven circulatory system including a thrombus, a continuous infusion apparatus and a filter;

FIG. 2 schematically depicts an arrangement of a thrombus and a single ultrasonic head;

FIG. 3 schematically depicts an arrangement a plurality of ultrasonic heads displaced around a thrombus;

FIG. 4 schematically depicts an arrangement of a two ultrasonic heads displaced angularly relative to a thrombus;

FIG. 5 schematically depicts an arrangement of a plurality of ultrasonic heads displaced linearly relative to a thrombus.

### DETAILED DESCRIPTION OF THE PREFERRED EMBODIMENTS

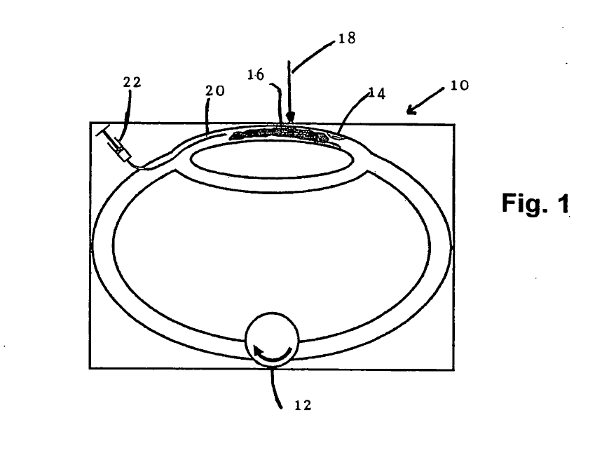

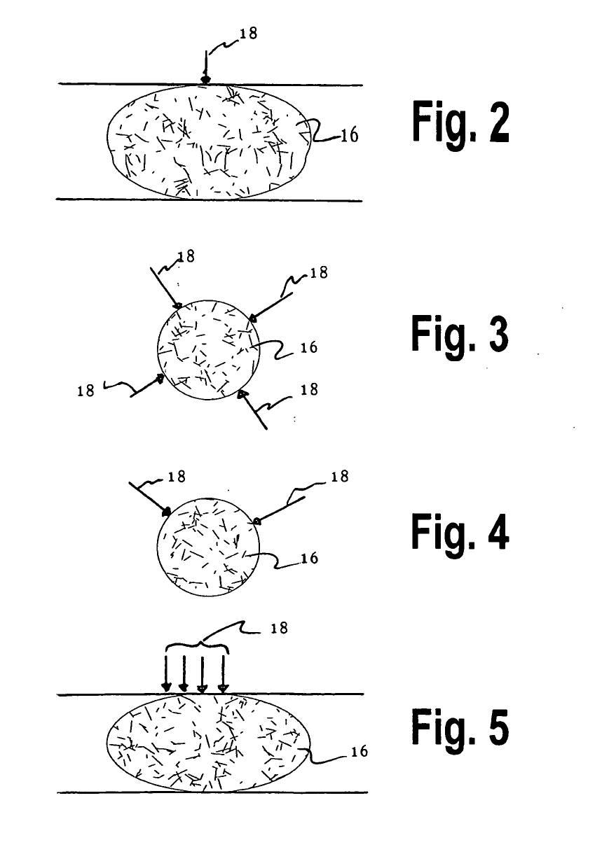

FIG. 1 represents a model of an arterial circulatory system 10 including a pump 12 for providing a pulsating flow of fluid therein. A pump found to be suitable for the experiments conducted in accordance with the invention is a Model KMP 2000 pump marketed by the Guerbet Company of Sulzbach, Germany. The pump 12 having a two-phased pressure profile, is ideally suited for simulating circulatory flow conditions and was set at a pumping rate of 300 ml/min and medium pressure of 100 mm Hg.

The by-pass used in the arterial models for the several experiments was a textile vascular graft of 6 mm diameter and 35 mm length of the kind used for arterial replacements or repair and is commercially available under the trademark Hemashield Gold®. Prior to the experiments and with a view to preventing the occurrence of air bubbles in and adjacent the wall of the grafts which might otherwise interfere with, or reflect, sonic energy, each graft was submersed in a 0.9% aqueous solution of NaCl for at least 48 hours. A standardized multipurpose filter 14 was attached to the output end of each graft to prevent the thrombus 16 from escaping from the bypass under the influence of the fluid pressure.

The thrombi used in the experiments were artificially prepared from 2.5 ml of whole blood free of any coagulation disorder and aged for 4 to 6 days. In each experiment, two individual thrombi were placed into the graft immediately upstream of the multipurpose filter 14.

While, in accordance with the invention, frequencies in the range from about 1 MHZ to about 20 MHZ are generally useful for treating thrombi, the experiments to be described were conducted at the common diagnostic frequency of 8.0 MHZ using a linear ultrasonic head 18. At a maximum length of 50 mm, the ultrasonic head 18 was generating sonic energy of 100 mW/cm². The source of sonic energy was a diagnostic apparatus (not shown) commercially available from Toshiba Corporation under the trademark ApplioXG®.

As will be apparent to those skilled in the art the experiments could have been conducted with greater efficiency by the use of more then one ultrasonic head. Several possible spatial arrangements of multiple heads have been shown in the drawings. Thus, a plurality of ultrasonic heads placed in parallel to a thrombus, or multiple heads placed so as to surround a thrombus. Another possible arrangement would require placing at least two ultrasound heads such that they would transit their sonic energy to a thrombus from different angles. In any of the arrangements the ultrasonic head or heads need not be stationary. Instead, they may be mounted for movement relative to a thrombus, either linearly and parallel thereto or in a radial or centripetal movement. Moreover, it is within the ambit of the invention to energize the ultrasound heads uniformly, at different levels, or even at changing levels.

In order to allow a local application of medication in front of a thrombus, a 4 F catheter 20 with a terminal opening was inserted into the graft upstream of the thrombus such that the tip of the catheter lay 10 mm forward of the thrombus. A continuous infusion pump 22 was connected to the catheter. Depending on the experiment, the pump 22 was fed with either 50 ml of a 0.9% aqueous NaCl solution or a dilution of 10 ml of ultrasound contrast agent in 40 ml of a 0.9% aqueous NaCl solution. The contrast agent was of the kind commercially available under the trademark SonoVue®. The rate of feed of the infusion pump 22 was always set at 50 ml/h.

The experiments were carried out in three categories of seven experiments each, and in each of the experiments the thrombolysis took place for about one hour:

| Category: | Ultrasound: | Application by Catheter |
|---|---|---|
| 1: −US; −CA; (Dry | Yes | .9% NaCl Solution |
| Run) | | |
| 2: +US; −CA | Yes | .9% NaCl Solution |
| 3: +US; +CA | Yes | .9% NaCl Solution & Contrast Agent |

(US = Ultrasound; CA = Contrast Agent)

Documenting took place by dissection of the graft and by photographing any residual thrombus.

The unmarked images were evaluated independently of each other to two observers and the quantity of the remainder of a thrombus was defined on the basis of a five-point-scale. (1: residue 0-20%; 2: residue 21-40%; 3: residue 41-60%; 4: residue 61-80%; 5: residue 81-100%). The images were compared against an image of a dissected non-lysed thrombus.

As expected, the experimental categories displayed thrombi of different degrees of lysing. The thrombi treated only with ultrasound were negligibly smaller than those treated without ultrasound and contrast agent. However, those thrombi affected by both ultrasound and contrast agent were lysed to a markedly greater extent. Moreover, in these instances the surface of the graft was noticeably devoid of micro thrombi adhering to the wall. The destruction of the microbubbles results in minute quantities of gas which rises to the surface in or ahead of the obstruction of the vain rather than being carried of, as they would be, in a open flow system.

The kappa concurrence between the two evaluating persons amounted to 0.662 and may thus be classified as substantial concurrence. Accordingly, for purposes of a further evaluation recourse was had to arithmetic mean values between the two evaluating persons. Based on the number of conducted experiments and the evaluation on a normalized scale level, non-parametric tests were used for comparing the categories. Nevertheless, analyzing the differences led to substantially the same results.

At median ranges of 15.14 (−US; −CA), 13.21 (+US; −CA) and 4.64 (+US; +CA) the Kruskal-Wallis-H test showed the existence of a most significant difference (p=0.001). A Mann-Whitney-U-test made it possible to establish the existence of a most significant difference (p=0.001) between categories “−US; −CA” and “+US; +CA” and a highly significant difference (p=0.009) between categories “+US; −CA” and “+US; +CA”. No statistically significant difference (p=0.620) could be established between categories “−US; −CA” and “+US; −CA”).

The categories of the experiments clearly demonstrated that in a dynamic model of an occluded arterial graft the application of ultrasound at the defined range and power level in combination with an ultrasound contrast agent offers an excellent potential for dissipating or lysing thrombi by means of the mechanical action of the components referred to supra. The remarkably lower number of micro thrombi at the surface of the grafts was especially noticeable and may be taken as an indication of the fact that such micro thrombi in particular may be lysed especially advantageously by a treatment of ultrasound and contrast agent.

Compared to the current state of the art in accordance with which ultrasound is usually applied at a low frequency range, i.e 10 kHz-1 MHz, and contrast agent is either not applied at all or intermittently only, the experiments described above not only indicate superior results but may also form a basis for clinically investigating likely synergistic effects with additional lysing medications. These may well lead to significantly reduced doses and durations of thrombolytic treatments coupled with a proportionately reduced rate of the side effects of conventional lysing therapy.

An additional benefit would be a reduced effective ischemia and, accordingly, the prevention or at least reduction of complications known to result from ischemia.

Those skilled in the art will appreciate the excellent potential of the results of the experiments set forth supra in terms of effective thrombolyses by the application of ultrasound in a diagnostic range as set forth above in combination with a contrast agent. It is believed that the instant invention in combination with the application of a lysing medication may well form the basis for significantly improved thrombolytic treatments of reduced duration and, therefore, a patient's exposure to such treatments.

What is claimed is:

1. A method of dissipating a thrombus, comprising the steps of applying to the thrombus at least one of an ultrasonic contrast agent and diagnostic ultrasound of a frequency in the range of from about 1 MHz to about 20 MHZ.

2. The method of claim 1, wherein the frequency of the ultrasound is 8 MHZ.

3. The method of claim 1, wherein the ultrasound is transmitted by at least one ultrasonic head.

4. The method of claim 3, wherein the ultrasound head is movable relative to the thrombus.

5. The method of claim 3, wherein the ultrasound is transmitted by at least two ultrasonic heads.

6. The method of claim 5, wherein the ultrasound heads are disposed angularly of each other in a plane intersecting with the thrombus.

7. The method of claim 5, wherein the ultrasound heads are movable relative to each other.

8. The method of claim 3, wherein the ultrasound is transmitted by a plurality of ultrasound heads disposed in a plane surrounding the thrombus.

9. The method of claim 8, wherein the ultrasound heads are movable relative to the thrombus.

10. The method of claim 3, wherein the ultrasound is transmitted by a plurality of ultrasound heads disposed in a plane extending along the axis of the thrombus.

11. The method of claim 10, wherein the ultrasound heads are movable relative to at least one of each other and the thrombus.

12. The method of claim 2, wherein the power of the ultrasound is 100 mW/cm².

13. The method of claim 12, wherein the power of the ultrasound is variable.

14. The method of claim 1, wherein the contrast agent is applied locally by a catheter.

15. The method of claim 1, wherein the contrast agent comprises gaseous microbubbles encasing sulfurhexafluoride.

16. The method of claim 1, wherein the contrast agent comprises gaseous microbubbles encasing dodecafluoropentane.
